(19) United States
(12) Patent Application Publication
(10) Pub. No.: US 2010/0130631 A1
(43) Pub. Date: May 27, 2010

(54) POLYESTER POYOL COMPOSITION FOR RIGID POLYURETHANE FOAMS,AND RIGID POLYURETHANE FOAM

(75) Inventors: Atsushi MIYATA, Ichihara-shi ,Chiba (JP); Tomoki TSUTSUI, Chiba-shi ,Chiba (JP); Nobuhito KOGA, Yokohama-shi ,Kanagawa (JP); Masahiro SASAKI, Ichihara-shi, Chiba (JP); Shinsuke MATSUMOTO, Ichihara-shi ,Chiba (JP); Kazuhiko OHKUBO, Yokiohama-shi ,Kanagawa (JP)

Correspondence Address: FOLEY AND LARDNER LLP;SUITE 500, 3000 K STREET NW, WASHINGTON, DC, 20007, US

(73) Assignee: Mitsui Chemicals ,Inc.

(21) Appl. No.: 12/451,328

(22) PCT Filed: Apr. 21, 2008
(86) PCT No.: PCT/JP2008/057679

§ 371 Date: Nov. 6, 2009

(30) Foreign Application Priority Data
May 11, 2007 (JP) 2007-127077

(51) Int. Cl. C08G 18/34; C07C 69/73

(52) U.S. Cl. 521/159; 560/181

(57) ABSTRACT

Polyester polyol compositions have a low viscosity and are suited for the production of rigid polyurethane foams. Compositions for rigid polyurethane foams contain the polyester polyol compositions. Rigid polyurethane foams are produced therefrom.

A polyester polyol composition is obtained by: a step (I) in which a dibasic acid compound (A) is reacted with a trihydric or higher polyhydric alcohol (a compound (B)); and a step (II) in which the reaction mixture from the step (I) is reacted with a hydroxycarboxylic acid compound (C); at least one compound (C) being a compound (C′) selected from the group consisting of ricinoleic acid compounds and 12-hydroxystearic acid compounds; the polyester polyol composition having a hydroxyl value in the range of 250 to 550 mg KOH/g and an average functionality of 3 to 8.

### FIELD OF THE INVENTION

The present invention relates to polyester polyol compositions, compositions for rigid polyurethane foams containing the same, and rigid polyurethane foams.

### BACKGROUND OF THE INVENTION

Rigid polyurethane foams have excellent thermal insulating properties, shaping properties and self-adhesiveness and are widely used as thermal insulating materials in refrigerators, freezers, cold storage warehouses and building panels. Chlorofluorocarbon was a conventional foaming agent for rigid polyurethane foams. From the aspects of protection of the ozone layer and prevention of the global warming, various chlorofluorocarbon-alternative foaming agents have been used.

Recent trends for environmental friendliness have led to increased demands for plant resins derived from plant resources as alternatives to petroleum resins from petroleum resources. Plant resins are produced from materials originating from plants that grow by photosynthesis absorbing CO₂ in air. They are carbon neutral, in other words, CO₂ released by incineration of the disposed resins is offset and is not added to the CO₂ emissions in air. The plant resins attract attention as materials contributing to the reduction of environmental burdens.

Patent Document 1 discloses a method for obtaining rigid polyurethane foams with use of a polyester polyol having an average functionality of 2.2 to 3.8 which is formed between an aliphatic polyhydric alcohol including a polyhydric alcohol of 3 or more functional groups, and a linear aliphatic polyvalent carboxylic acid of 2 or more carbon atoms.

According to Patent Document 1, however, hydrochlorofluorocarbons and hydrofluorocarbons are used as foaming agents for rigid polyurethane foams. The hydrochlorofluorocarbons are ozone depleting substances although weaker than chlorofluorocarbon and have a high global warming potential, and therefore the use thereof causes heavy environmental burdens. The hydrofluorocarbons do not have ozone destroying capability but do have a high global warming potential, causing heavy environmental loads.

Patent Document 2 discloses fatty acid-modified polyester polyol compositions for rigid urethane foams that are obtained by reacting a polybasic acid and a polyhydric alcohol in the presence of at least one selected from a fatty acid of 8 to 30 carbon atoms and a fat/oil or an aliphatic compound containing the fatty acid. Patent Document 2 is concerned with obtaining polyester polyols having high compatibility with chlorofluorocarbon, and does not disclose any foaming agents other than chlorofluorocarbon. The working examples only disclose aromatic polybasic acids as the polybasic acids, and the polyester polyols obtained have a very high viscosity, with even the lowest viscosity being 9120 cps at 25° C. Accordingly, shaping the compositions into polyurethane foams is difficult.

Patent Document 3 describes that a polymerized vegetable oil/fat is epoxidized and is thereafter ring-opened with a monohydric alcohol into a hydroxyalkoxy compound for use as a polyol for rigid polyurethane foams. The polyols synthesized according to Patent Document 3, however, have a maximum hydroxyl value of only 108 mg KOH/g and are excessively polymerized for use in general rigid polyurethane foams. Foams obtainable therewith are soft and do not show hardness required for rigid foams.

Patent Document 4 discloses rigid polyurethane foams obtainable using a polyester polyol formed between diglycerol and a polyvalent carboxylic acid of 2 or more carbon atoms, and water as a foaming agent. However, the polyester polyols obtained in Patent Document 4 have a viscosity of 30,000 mPa·s or more, and the foaming involves conventional petroleum-derived materials, failing to contribute to reduced environmental burdens.
Patent Document 1: Japan Patent No. 3651038
Patent Document 2: Japan Patent No. 3197508
Patent Document 3: JP-A-2006-1865
Patent Document 4: JP-A-2005-206691

### SUMMARY OF THE INVENTION

Objects of the present invention are to provide polyester polyol compositions that have a low viscosity and are suited for the production of rigid polyurethane foams, and to provide compositions for rigid polyurethane foams containing the polyester polyol compositions, and rigid polyurethane foams.

The present inventors studied diligently to achieve the above objects and have invented specific polyester polyol compositions. The compositions have been found to have a low viscosity and be suited for the production of rigid polyurethane foams. It has been further found that compositions containing the polyester polyol compositions can give rigid polyurethane foams that are widely used as thermal insulating materials in refrigerators, freezers, cold storage warehouses and building panels. The present invention has been completed based on the findings.

A polyester polyol composition according to the present invention is obtained by:

a step (I) in which at least one compound (A) selected from the group consisting of dibasic acids, alkyl esters formed between a C1-6 monohydric saturated alcohol and a dibasic acid, and alkenyl esters formed between a C3-6 monohydric unsaturated alcohol with one carbon-carbon double bond and a dibasic acid, is reacted with a trihydric or higher polyhydric alcohol (a compound (B)); and

a step (II) in which the reaction mixture from the step (I) is reacted with at least one compound (C) selected from the group consisting of hydroxycarboxylic acids, alkyl esters formed between a C1-6 monohydric saturated alcohol and a hydroxycarboxylic acid, and alkenyl esters formed between a C3-6 monohydric unsaturated alcohol with one carbon-carbon double bond and a hydroxycarboxylic acid;

at least one compound (C) being a compound (C′) selected from the group consisting of ricinoleic acid, alkyl esters formed between a C1-6 monohydric saturated alcohol and ricinoleic acid, alkenyl esters formed between a C3-6 monohydric unsaturated alcohol with one carbon-carbon double bond and ricinoleic acid, 12-hydroxystearic acid, alkyl esters formed between a C1-6 monohydric saturated alcohol and 12-hydroxystearic acid, and alkenyl esters formed between a C3-6 monohydric unsaturated alcohol with one carbon-carbon double bond and 12-hydroxystearic acid;

the polyester polyol composition having a hydroxyl value in the range of 250 to 550 mg KOH/g and an average functionality of 3 to 8.

A polyester polyol composition according to the present invention is obtained by:

a step (1) of reacting at least one compound (A) selected from the group consisting of dibasic acids, alkyl esters formed between a C1-6 monohydric saturated alcohol and a dibasic acid, and alkenyl esters formed between a C3-6 monohydric unsaturated alcohol with one carbon-carbon double bond and a dibasic acid;

a trihydric or higher polyhydric alcohol (a compound (B)); and

at least one compound (C) selected from the group consisting of hydroxycarboxylic acids, alkyl esters formed between a C1-6 monohydric saturated alcohol and a hydroxycarboxylic acid, and alkenyl esters formed between a C3-6 monohydric unsaturated alcohol with one carbon-carbon double bond and a hydroxycarboxylic acid;

at least one compound (C) being a compound (C′) selected from the group consisting of ricinoleic acid, alkyl esters formed between a C1-6 monohydric saturated alcohol and ricinoleic acid, alkenyl esters formed between a C3-6 monohydric unsaturated alcohol with one carbon-carbon double bond and ricinoleic acid, 12-hydroxystearic acid, alkyl esters formed between a C1-6 monohydric saturated alcohol and 12-hydroxystearic acid, and alkenyl esters formed between a C3-6 monohydric unsaturated alcohol with one carbon-carbon double bond and 12-hydroxystearic acid;

the polyester polyol composition having a hydroxyl value in the range of 250 to 550 mg KOH/g and an average functionality of 3 to 8.

The polyester polyol compositions are preferably obtained using 2 to 70 parts by weight of the compound (A), 10 to 85 parts by weight of the compound (B), and 5 to 85 parts by weight of the compound (C) (the total of the compounds (A), (B) and (C) is 100 parts by weight).

The compound (C′) preferably accounts for 10 to 100 wt % of 100 wt % of the compound(s) (C).

In the polyester polyol compositions, the compound (A), the compound (B) and the compound (C) are each preferably a plant-derived compound.

In an aspect of the invention, a composition for rigid polyurethane foams comprises the polyester polyol composition, a polyisocyanate, at least one foaming agent selected from the group consisting of water and hydrocarbon compounds, and a catalyst.

In an aspect of the invention, a rigid polyurethane foam is formed from the composition for rigid polyurethane foams.

### ADVANTAGEOUS EFFECTS OF THE INVENTION

The polyester polyol compositions of the invention have a low viscosity and have an appropriate hydroxyl value and an appropriate functionality for use in compositions for rigid polyurethane foams and in rigid polyurethane foams formed from the compositions.

If a fatty acid having no hydroxyl groups is used, the functionality becomes smaller with increasing weight of the fatty acid. By using the compounds (C), the hydroxyl value can be controlled as desired without reducing the number of functional groups.

The alcohols used in the polyester polyol compositions of the invention are not dihydric alcohols but are trihydric or higher polyhydric alcohols. As a result, the polyester polyol compositions will not contain dihydric polyester polyols, and the compositions for rigid polyurethane foams that contain the polyester polyol compositions can be sufficiently crosslinked at foaming and will provide rigid polyurethane foams having excellent dimensional stability.

The compounds (C) such as ricinoleic acid, 12-hydroxystearic acid, alkyl esters thereof and alkenyl esters thereof have long carbon chains and thus allow for the reduction in viscosity of the obtainable polyester polyol compositions.

Because of these advantageous properties of the polyester polyol compositions, rigid polyurethane foams can be prepared using at least one foaming agent selected from water and hydrocarbon compounds. Thus, the rigid polyurethane foams of the invention contribute to the reduction of environmental burdens. When the compounds (A), (B) and (C) are plant-derived compounds, the rigid polyurethane foams contribute to reduced CO₂ emissions and reduced environmental burdens. The rigid polyurethane foams are usable as thermal insulating materials in building panels, refrigerators, freezers and pipes, and as structural supports in housings and vehicles.

### PREFERRED EMBODIMENTS FOR CARRYING OUT THE INVENTION

The polyester polyol compositions, the compositions for rigid polyurethane foams, and the rigid polyurethane foams according to the present invention will be described in detail hereinbelow.

### <Polyester Polyol Compositions>

The polyester polyol compositions according to the present invention may be obtained by: a step (I) in which at least one compound (A) selected from the group consisting of dibasic acids, alkyl esters formed between a C1-6 monohydric saturated alcohol and a dibasic acid, and alkenyl esters formed between a C3-6 monohydric unsaturated alcohol with one carbon-carbon double bond and a dibasic acid, is reacted with a trihydric or higher polyhydric alcohol (a compound (B)); and a step (II) in which the reaction mixture from the step (I) is reacted with at least one compound (C) selected from the group consisting of hydroxycarboxylic acids, alkyl esters formed between a C1-6 monohydric saturated alcohol and a hydroxycarboxylic acid, and alkenyl esters formed between a C3-6 monohydric unsaturated alcohol with one carbon-carbon double bond and a hydroxycarboxylic acid.

Alternatively, the polyester polyol compositions according to the present invention may be obtained by a step (1) of reacting at least one compound (A) selected from the group consisting of dibasic acids, alkyl esters formed between a C1-6 monohydric saturated alcohol and a dibasic acid, and alkenyl esters formed between a C3-6 monohydric unsaturated alcohol with one carbon-carbon double bond and a dibasic acid; a trihydric or higher polyhydric alcohol (a compound (B)); and at least one compound (C) selected from the group consisting of hydroxycarboxylic acids, alkyl esters formed between a C1-6 monohydric saturated alcohol and a hydroxycarboxylic acid, and alkenyl esters formed between a C3-6 monohydric unsaturated alcohol with one carbon-carbon double bond and a hydroxycarboxylic acid.

In the polyester polyol compositions, the at least one compound (C) includes at least one compound (C′) selected from the group consisting of ricinoleic acid, alkyl esters formed between a C1-6 monohydric saturated alcohol and ricinoleic acid, alkenyl esters formed between a C3-6 monohydric unsaturated alcohol with one carbon-carbon double bond and ricinoleic acid, 12-hydroxystearic acid, alkyl esters formed between a C1-6 monohydric saturated alcohol and 12-hydroxystearic acid, and alkenyl esters formed between a C3-6 monohydric unsaturated alcohol with one carbon-carbon double bond and 12-hydroxystearic acid.

The polyester polyol compositions have a hydroxyl value in the range of 250 to 550 mg KOH/g, preferably 300 to 500 mg KOH/g, and an average functionality of 3 to 8, preferably 3 to 6.

The polyester polyol compositions are preferably obtained by reacting 2 to 70 parts by weight of the compound (A), 10 to 85 parts by weight of the compound (B), and 5 to 85 parts by weight of the compound (C). The compounds (A), (B) and (C) are preferably plant-derived compounds.

The polyester polyol compositions usually have a viscosity at 25° C. of not more than 20000 mPa·s and are useful for the production of rigid polyurethane foams.

### [Compounds (A)]

Examples of the compounds (A) include dibasic acids, alkyl esters formed between a C1-6 monohydric saturated alcohol and a dibasic acid, and alkenyl esters formed between a C3-6 monohydric unsaturated alcohol with one carbon-carbon double bond and a dibasic acid.

The dibasic acids are not particularly limited as long as they have two carboxyl groups in the molecule. Examples thereof include oxalic acid, malonic acid, succinic acid, glutaric acid, adipic acid, pimelic acid, suberic acid, sebacic acid, azelaic acid, undecanoic diacid, dodecanoic diacid, tridecanoic diacid, tetradecanoic diacid, pentadecanoic diacid, octadecanoic diacid, nonadecanoic diacid, eicosanoic diacid, methylhexanoic diacid, dimer acid, maleic acid, fumaric acid, itaconic acid and citraconic acid. The dibasic acids may be used singly, or two or more kinds may be used in combination.

The compounds (A) may be derivatives from the above dibasic acids. That is, the compounds (A) may be alkyl esters formed between a C1-6-monohydric saturated alcohol and the dibasic acid, and alkenyl esters formed between a C3-6 monohydric unsaturated alcohol with one carbon-carbon double bond and the dibasic acid.

These esters may be monoesters or diesters. To achieve higher reactivity, methyl esters and ethyl esters are preferable.

From the aspect of reduced environmental burdens, it is preferable to use as the compound (A) at least one plant-derived dibasic acid such as sebacic acid, azelaic acid, dimer acid or itaconic acid, or at least one alkyl ester or alkenyl ester derived from the plant-derived dibasic acid.

### [Trihydric or Higher Polyhydric Alcohols (Compounds (B)]

The trihydric or higher polyhydric alcohols (compounds (B)) are not particularly limited as long as they have three or more hydroxyl groups in the molecule. Examples thereof include glycerol, trimethylolpropane, diglycerol, triglycerol, erythritol, pentaerythritol, sorbitol, monosaccharides such as glucose, and disaccharides such as sucrose and trehalose.

The compounds (B) maybe used singly, or two or more kinds maybe used in combination. To reduce environmental burdens, it is preferable to use one, or two or more kinds of plant-derived compounds selected from glycerol, erythritol, sorbitol, monosaccharides such as glucose, and disaccharides such as sucrose and trehalose.

### [Compounds (C)]

Examples of the compounds (C) include hydroxycarboxylic acids, alkyl esters formed between a C1-6 monohydric saturated alcohol and a hydroxycarboxylic acid, and alkenyl esters formed between a C3-6 monohydric unsaturated alcohol with one carbon-carbon double bond and a hydroxycarboxylic acid.

As the compound (C), at least one compound (C′) is used which is selected from ricinoleic acid, alkyl esters formed between a C1-6 monohydric saturated alcohol and ricinoleic acid, alkenyl esters formed between a C3-6 monohydric unsaturated alcohol with one carbon-carbon double bond and ricinoleic acid, 12-hydroxystearic acid, alkyl esters formed between a C1-6 monohydric saturated alcohol and 12-hydroxystearic acid, and alkenyl esters formed between a C3-6 monohydric unsaturated alcohol with one carbon-carbon double bond and 12-hydroxystearic acid.

Examples of the compounds (C′) other than ricinoleic acid and 12-hydroxystearic acid include methyl ricinoleate, ethyl ricinoleate, butyl ricinoleate, methyl 12-hydroxystearate and ethyl 12-hydroxystearate.

It is preferable to use at least one compound (C′) selected from ricinoleic acid, alkyl esters formed between a C1-6 monohydric saturated alcohol and ricinoleic acid, and alkenyl esters formed between a C3-6 monohydric unsaturated alcohol with one carbon-carbon double bond and ricinoleic acid. These compounds have an unsaturated bond in the fatty acid moiety, and the use thereof tends to lower the viscosity of the obtainable polyester polyol compositions.

The compounds (C′) maybe used singly, or two or more kinds may be used in combination. To achieve higher reactivity, ricinoleic acid, methyl ricinoleate, ethyl ricinoleate, 12-hydroxystearic acid, methyl 12-hydroxystearate and ethyl 12-hydroxystearate are preferable.

By the use of the compounds (C′), the polyester polyol compositions of the invention have a lower viscosity compared to the polyester polyol compositions described in Japan Patent No. 3197508. As a result, enhanced stirring efficiency is achieved in the manufacturing of rigid polyurethane foams from compositions containing the polyester polyol compositions, and homogeneous foams are easily obtained.

In addition to the compounds (C′), hydroxycarboxylic acids may be used as the compounds (C) without limitation as long as they have one hydroxyl group and one carboxyl group in the molecule. Examples thereof include lactic acid, hydroxyacetic acid, 3-hydroxybutyric acid, hydroxyisocaproic acid, 2-hydroxyhexanoic acid, 3-hydroxyhexanoic acid, 2-hydroxy-3-methylpentanoic acid, 2-hydroxy-4-methylvaleric acid, deoxymevalonic acid and 2-hydroxyoctanoic acid. These acids may be used singly, or two or more kinds may be used in combination.

To reduce environmental burdens, lactic acid produced from plants is a preferred hydroxycarboxylic acid for use as the compound (C) together with the compounds (C′).

Derivatives of the hydroxycarboxylic acids may also be used, with examples including alkyl esters formed between a C1-6 monohydric saturated alcohol and the hydroxycarboxylic acid, and alkenyl esters formed between a C3-6 monohydric unsaturated alcohol with one carbon-carbon double bond and the hydroxycarboxylic acid.

Specific examples of such esters include methyl lactate, methyl hydroxyacetate and methyl 3-hydroxybutyrate.

The compound (C′) usually accounts for 10 to 100 wt %, and preferably 50 to 100 wt % of 100 wt % of the compounds (C).

Below the lower limit, the amount of the compounds (C′) is insufficient and the viscosity of the polyester polyol compositions may not be lowered.

The polyester polyol compositions may be obtained by reacting the compounds (A), (B) and (C). The quantitative ratio of the compounds (A) and (B) is not particularly limited as long as the obtainable polyester polyol has a hydroxyl value of 250 to 550 mg KOH/g and a functionality of 3 to 8. Generally, the compositions may be obtained by reacting 2 to 70 parts by weight of the compound (A), 10 to 85 parts by weight of the compound (B) and 5 to 85 parts by weight of the compound (C), and preferably by reacting 10 to 60 parts by weight of the compound (A), 15 to 60 parts by weight of the compound (B) and 10 to 70 parts by weight of the compound (C), based on 100 parts by weight of the total of the compounds (A), (B) and (C).

If the amount of the compound (A) is excessively small, the functionality may be smaller than the desired level. If the amount thereof is excessively large, the hydroxyl value may be lowered or the number of functional groups may be excessively increased to result in a very viscous polyester polyol. If the amount of the compound (B) is excessively small, the hydroxyl value may be excessively low. If the amount thereof is excessively large, a large amount of the compound (A) may remain unreacted in the polyester polyol and may be separated from the polyester polyol. If the amount of the compound (C) is excessively small, the viscosity may not be lowered as desired. If the amount thereof is excessively large, the hydroxyl value of the polyester polyol may be excessively low or the obtainable polyester polyol may not have a sufficient functionality.

### [Processes for Producing Polyester Polyol Compositions]

The polyester polyol compositions may be produced by known esterification methods. For example, the polyester polyol compositions may be produced by a step (I) of reacting the compound (A) and the compound (B), and a step (II) of reacting the reaction mixture from the step (I) and the compound (C). Alternatively, the polyester polyol compositions may be prepared by a step (1) of reacting the compound (A), the compound (B) and the compound (C).

In detail, a two-stage reaction may be adopted in which the compound (A) and the compound (B) are condensed together and the compound (C) is added to the reaction mixture and is dehydrated and condensed together. Alternatively, a one-stage reaction may be used in which the compounds (A), (B) and (C) are collectively dehydrated and condensed.

For example, the dehydration condensation may be carried out at a high temperature without a solvent under an atmosphere of an inert gas such as nitrogen. Other known methods such as solution polymerization may be used. In the high-temperature, solvent-free condensation, the temperature is not particularly limited as long as dehydration condensation takes place. In view of decomposition and volatilization of materials, the temperature is preferably in the range of 140 to 250° C., and more preferably 160 to 230° C. The reaction pressure may be atmospheric pressure, elevated pressure or reduced pressure. In view of reaction efficiency, atmospheric pressure or reduced pressure is preferable. Esterification catalysts may be used, with examples including tin catalysts such as tin octylate and dibutyltin dilaurate and other catalysts such as titanium catalysts.

The hydroxyl value and the functionality of the polyester polyol compositions may be controlled by adjusting the amounts of the compounds (A), (B) and (C).

The hydroxyl value may be obtained by determining the number of hydroxyl groups contained per 1 g of the polyester polyol composition from the reaction between the hydroxyl groups and the carboxylic acids in the compounds (A), (B) and (C). In detail, the number of hydroxyl groups per 1 g of the polyester polyol composition may be determined according to Equation (1) below.

[Formula 1]

Number of hydroxyl groups per 1 g of polyester polyol composition=[{number of hydroxyl groups contained in compounds (B) and (C)}−{number of carboxyl groups contained in compounds (A) and (C)}]/{weight (g) of compound (A)+weight (g) of compound (B)+weight (g) of compound (C)−weight (g) of condensation water} (1)

The weight of condensation water may be determined by collecting the condensation water from the water separation apparatus into a water receiver and measuring the weight thereof upon completion of the reaction.

The number of functional groups (per molecule) of the polyester polyol, composition may be determined from Equations (I) to (III).

[Formula 2]

Number of functional groups per molecule=number of hydroxyl groups per 100 g of reaction product/number of molecules per 100 g of reaction product (I)

Number of hydroxyl groups per 100 g of reaction product=100×hydroxyl value of reaction product/56108 (II)

Number of molecules per 100 g of reaction product=100/(amount (g) of monomers−condensation water (g))×(number of moles of polyhydric alcohol−number of moles of dibasic acid) (III)

The condensation water in Equations (1) and (III) is byproduced water in the production of the polyester polyol composition in the case where the compounds (A) and (C) are not the alkyl esters or the alkenyl esters, but the compound (A) is a dibasic acid and the compound (C) is a hydroxycarboxylic acid.

When the alkyl esters or the alkenyl esters are used as the compounds (A) or a part of the compounds (C), a corresponding alcohol and water are byproduced in the production of the polyester polyol composition. In this case, the number of hydroxyl groups per 1 g of the polyester polyol composition and the number of functional groups of the polyester polyol composition may be determined by substituting the condensation water in Equations (1) and (III) by the total of the byproduced alcohol and water.

### (Compositions for Rigid Polyurethane Foams)

The compositions for rigid polyurethane foams contain the polyester polyol composition, a polyisocyanate, at least one foaming agent selected from water and hydrocarbon compounds, and a catalyst.

The compositions for rigid polyurethane foams may further contain foam stabilizers or other additives.

The compositions for rigid polyurethane foams may be generally obtained by mixing the above-mentioned components. Preferably, the compositions for rigid polyurethane foams are prepared immediately before use.

In a preferred embodiment, a resin premix is prepared beforehand which contains the polyester polyol composition, a foaming agent and a catalyst and optionally a foam stabilizer and other additive, and a polyisocyanate is mixed therewith immediately before use to give a composition for rigid polyurethane foams.

The polyester polyol compositions used in the compositions for rigid polyurethane foams are as described hereinabove. The other components are as described hereinbelow.

### [Polyisocyanates]

Known polyisocyanates may be used in the compositions for rigid polyurethane foams according to the present invention. Examples include toluene diisocyanates (hereinafter, TDI) and diphenylmethane diisocyanates (hereinafter, MDI).

Examples of TDI include 100% isomerically pure 2,4-TDI, an isomeric mixture containing 2,4-TDI/2,6-TDI (weight ratio)=80/20, and an isomeric mixture containing 2,4-TDI/2,6-TDI (weight ratio)=65/35.

Examples of MDI include compounds based on 4,4′-MDI, and polymeric MDI containing polynuclear molecules with three or more nuclei (e.g., COSMONATE series manufactured by MITSUI CHEMICALS POLYURETHANES INC.).

Examples of the organic polyisocyanate compounds further include modified polyisocyanate compounds such as polyisocyanurates, carbodiimide-modified polyisocyanates, prepolymerized polyisocyanates (prepolymers obtained from the organic polyisocyanates and active hydrogen-containing compounds and having an isocyanate group at a molecular end) and urethodione-modified polyisocyanates. The organic polyisocyanate compounds and the modified products thereof may be used singly, or two or more kinds may be used in combination.

The amount of the polyisocyanate to be mixed with the resin premix may be determined based on the ratio between the isocyanate groups in the polyisocyanate and the active hydrogen in the resin premix (NCO/H (the equivalent ratio of active hydrogen)). The ratio is desirably in the range of 0.7 to 2.0, preferably 0.9 to 1.6, and more preferably 1.0 to 1.4. If the ratio is below 0.7, crosslinking may not proceed sufficiently and the obtainable foams may not have properties such as hardness required for rigid polyurethane foams. If the ratio exceeds 2.0, the proportion of the plant-derived materials in the foams is excessively low.

### [Foaming Agents]

Examples of the foaming agents in the compositions for rigid polyurethane foams include water and hydrocarbon compounds.

Water and hydrocarbon compounds have lower ozone destroying capability compared to the conventionally used chlorofluorocarbons, hydrochlorofluorocarbons and hydrofluorocarbons and also have low global warming potential.

Physical foaming agents and chemical foaming agents may be used. They may be used singly, or a plurality thereof may be used in combination.

Specific examples of the chemical foaming agents include water. The chemical foaming agents are preferably used in an amount of 0.5 to less than 15.0 parts by weight based on 100 parts by weight of the polyester polyol composition.

Examples of the physical foaming agents include hydrocarbon compounds such as cyclopentane, iso-pentane, n-pentane, n-hexane, 2,2-dimethylbutane, 2,3-dimethylbutane, 2-methylpentane and 3-methylpentane. They may be used singly, or a plurality thereof may be used in combination. The amount of the physical foaming agents maybe determined appropriately depending on use of the foams, but is preferably in the range of 6 to 40 parts by weight based on 100 parts by weight of the polyester polyol composition.

### [Catalysts]

Any catalysts generally used in the manufacturing of urethane foams may be used in the compositions for rigid polyurethane foams. Examples include, but are not limited to, amines, aziridines, quaternary ammonium compounds, alkali metal salts, tin compounds, alcoholate compounds, phenolate compounds, metal halides and metal complex compounds.

The amines include trimethylaminoethylpiperazine, triethylamine, tripropylamine, N-methylmorpholine, N-ethylmorpholine, triethylenediamine, tetramethylhexamethylenediamine, dimethylcyclohexylamine, diazobicycloundecene, bis(2-dimethylaminoethyl)ether and 1,3,5-tris(dimethylaminopropyl)hexahydro-s-triazine.

The aziridines include 2-ethylaziridine.

The quaternary ammonium compounds include carboxylates of tertiary amines. The alkali metal salts include potassium octylate and sodium acetate.

Zinc compounds such as zinc naphthenate and zinc octylate may be used. The tin compounds include dibutyltin diacetate and dibutyltin dilaurate. The alcoholate compounds include sodium methoxide and sodium ethoxide.

The phenolate compounds include potassium phenoxide, lithium phenoxide and sodium phenoxide. The metal halides include iron chloride, zinc chloride, zinc bromide and tin chloride. The metal complex compounds include acetylacetone metal salts.

The catalysts may be used singly, or two or more kinds may be used in combination. The amount of the catalysts is preferably in the range of 0.01 to 10.0 parts by weight, and more preferably 0.1 to 2 parts by weight based on 100 parts by weight of the polyester polyol composition.

### [Foam Stabilizers]

The compositions for rigid polyurethane foams usually contain a foam stabilizer. The foam stabilizers used in the invention may be conventional silicon-containing organic surfactants such as silicone derivatives (alkylene oxide-modified polydimethylsiloxanes terminated with an alkoxyl group or an active OH group). Examples further include nonionic surfactants such as polyoxyethylene octadecylamine and long-chain fatty acid alkylolamides.

Specific examples are SF-2935F, SF-2938F, SF-2940F, SF-2945F, SF-2908, SRX-294A, SH-190, SH-192, SH-193, SZ-1127, SZ-1142, SZ-1605, SZ-1627, SZ-1629, SZ-1642, SZ-1645, SZ-1646, SZ-1653, SZ-1675, SZ-1694, SZ-1708, SZ-1711, L-580, L-5420, L-5440 and L-5740 manufactured by Toray Dow Corning Silicone Co., Ltd.; Tegostab B-8461, B-8462, B-8466, B-8467, B-8474 and B-8481 manufactured by TH. Gold Schmit AG; and X-20-1328, F-388,

F-394, F-327, F-345 and F-305 manufactured by Shin-Etsu Chemical Co., Ltd.

In a preferred embodiment, the compositions for rigid polyurethane foams contain the foam stabilizer. The foam stabilizers may be used singly, or two or more kinds may be used in combination. The amount thereof is preferably in the range of 0.5 to 10.0 parts by mass, more preferably 0.5 to 5.0 parts by mass, and still more preferably 1.0 to 3.0 parts by mass based on 100 parts by weight of the polyester polyol composition.

### [Additives]

In the invention, various additives may be used depending on uses or purposes. Examples of the additives include flame retardants, antioxidants, coloring agents, plasticizers, stabilizers, compatibilizers and cell-opening agents.

### [Rigid Polyurethane Foams]

Rigid polyurethane foams may be obtained from the compositions for rigid polyurethane foams of the invention.

The rigid polyurethane foams have excellent dimensional stability and are usable in thermal insulating materials such as building panels, refrigerators, freezers and pipes, and structural supports such as housings and vehicles.

The rigid polyurethane foams usually have a dimensional change of not more than 3%, and preferably not more than 2%. In detail, this dimensional change is determined as follows. The rigid polyurethane foam is cut approximately to 80×80×40 mm, and the size is measured with a vernier caliper. The pieces are allowed to stand in ovens at 70° C., −30°, or 70° C. and 95% humidity. After 72 hours in the ovens, the size of the pieces is measured. The average of changes in length, width and height is obtained as the dimensional change.

In a preferred embodiment of the preparation of rigid polyurethane foams, the polyester polyol composition, the foaming agent and the catalyst optionally together with the foam stabilizer and other additives are mixed together to give a resin premix, the resin premix is contacted with the polyisocyanate into a composition, and rigid polyurethane foams are obtained from the composition.

It is preferable that the resin premix and the polyisocyanate are mixed together immediately before the foaming process. They may be mixed by a dynamic mixing method, a static mixing method, or both. Exemplary dynamic mixing methods include mixing by a stirring blade. Exemplary static mixing methods include mixing in a machine head mixing chamber of a foaming device, and mixing in a feed pipe with a static mixer. The mixing time is usually not more than 60 seconds.

Herein, the ratio between the isocyanate groups in the polyisocyanate and the active hydrogen in the resin premix (NCO/H (the equivalent ratio of active hydrogen)) is desirably in the range of 0.7 to 2.0, preferably 0.9 to 1.6, and more preferably 1.0 to 1.4. If the ratio is below 0.7, crosslinking may not proceed sufficiently and the obtainable foams may not have properties such as hardness required for rigid polyurethane foams. If the ratio exceeds 2.0, the proportion of the plant-derived materials in the foams is excessively low.

The mixing temperature may be determined appropriately depending on the desired quality of rigid polyurethane foams, or kinds and amounts of the materials. For example, the liquid temperature in the mixing of the resin premix and the polyisocyanate is preferably from 15 to 50° C.

The mixture liquid may be foamed using an open mold or a closed mold. The materials of the molds are not particularly limited, but materials such as metals are preferable because the temperature may be controlled easily. When the mold temperature is controlled, the mold temperature is preferably in the range of 30 to 70° C., and more preferably 35 to 60° C.

### EXAMPLES

The present invention will be described based on examples hereinbelow without limiting the scope of the invention.

In Examples, parts indicate parts by weight. The amounts of components in Table 1 are expressed in parts by weight. In Examples and Comparative Examples, analysis or measurements were carried out by the following methods.

### <Properties of Polyester Polyol Compositions>

### (1) Hydroxyl Value

The hydroxyl value of the polyester polyol compositions was determined by the method described in JIS K-6901, Section 5.4.

### (2) Acid Value

The acid value of the polyester polyol compositions was determined by the method described in JIS K-6901, Section 5.3.

### (3) Functionality

The number of functional groups (per molecule) was determined from Equations (I) to (III) below.

[Formula 3]

Number of functional groups per molecule=number of hydroxyl groups per 100 g of reaction product/number of molecules per 100 g of reaction product (I)

Number of hydroxyl groups per 100 g of reaction product=100×hydroxyl value of reaction product/56108 (II)

Number of molecules per 100 g of reaction product=100/(amount (g) of monomers−condensation water (g))×(number of moles of polyhydric alcohol−number of moles of dibasic acid) (III)

### (4) Viscosity

The viscosity was measured at 25° C. with an E-type viscometer (VISCOMETER TV-30, manufactured by TOKI SANGYO CO., LTD.)

### <Properties of Rigid Polyurethane Foams>

### (5) Core Density

The core density was measured by the method described in JIS K-6400. The core density is an apparent density according to Japanese Industrial Standards (JIS). In the invention, rectangular foam samples cut from a foam sample were tested.

### (6) Compressive Strength

In accordance with JIS K-7220, Rigid cellular plastics—Determination of compression properties, a foam was cut to 80×80×40 mm and was compressed to determine the compressive strength in a direction parallel to the expansion direction.

### (7) Flexural Strength

In accordance with JIS K-7221-2, Rigid cellar plastics—Determination of flexural properties—Part 2: Determination of flexural strength and apparent flexural modulus of elasticity, a foam was cut to 150×25×20 mm and was tested to determine the compressive strength in a direction parallel to the expansion direction.

### (8) Dimensional Stability

A foam was cut approximately to 80×80×40 mm, and the size was measured with a vernier caliper. The pieces were allowed to stand in ovens at 70° C., −30°, or 70° C. and 95% humidity. After 72 hours in the ovens, the size of the pieces was measured. The average of changes in length, width and height was recorded as the dimensional change.

For example, when the pieces having been allowed to stand under the above conditions have a size of 79×78×39 mm, the dimensional change is calculated to be −2.1% from Equations (2) to (5) below.

[Formula 4]

(79-80)/80×100=−1.25% (2)

(78-80)/80×100=−2.5% (3)

(39-40)/40×100=−2.5% (4)

{(−1.25)+(−2.5)+(−2.5)}/3=−2.0833 . . . ≈−2.1% (5)

### (9) Closed Cell Percentage

In accordance with ASTM D 2856, a foam was cut to 35×25×25 mm and was analyzed with AccuPyc 1330 manufactured by Shimadzu Corporation to determine the closed cell percentage.

### Example 1

### (Synthesis of Polyester Polyol Composition (1))

A polymerization vessel equipped with a reflux condenser, a water separator, a nitrogen gas inlet tube, a thermometer and a stirrer was charged with 344.32 parts of glycerol and 397.01 parts of sebacic acid, and a dehydration reaction was performed at 180° C. After the reaction was confirmed to have proceeded to an extent such that the acid value of the reaction product was not more than 3, 350.92 parts of ricinoleic acid (CO-FA manufactured by ITOH OIL CHEMICALS CO., LTD.) was added and a dehydration reaction was conducted at 180° C. When the acid value of the reaction product was confirmed to be not more than 2, the reaction product was withdrawn from the polymerization vessel and was cooled to give a polyester polyol composition (1).

The polyester polyol had a hydroxyl value of 385 mg KOH/g, an acid value of 1.9 mg KOH/g, a viscosity of 5780 mPa·s, and a functionality of 4. The condensation yielded 92.25 parts of water.

### Example 2

### (Synthesis of Polyester Polyol Composition (2))

A polymerization vessel equipped with a reflux condenser, a water separator, a nitrogen gas inlet tube, a thermometer and a stirrer was charged with 420.9 parts of glycerol and 473.91 parts of sebacic acid, and a dehydration reaction was performed at 180° C. After the reaction was confirmed to have proceeded to an extent such that the acid value of the reaction product was not more than 3, 201.99 parts of ricinoleic acid (CO-FA manufactured by ITOH OIL CHEMICALS CO., LTD.) was added and a dehydration reaction was conducted at 180° C. When the acid value of the reaction product was confirmed to be not more than 2, the reaction product was withdrawn from the polymerization vessel and was cooled to give a polyester polyol composition (2).

The polyester polyol had a hydroxyl value of 485 mg KOH/g, an acid value of 1.9 mg KOH/g, a viscosity of 7100 mPa·s, and a functionality of 4. The condensation yielded 96.8 parts of water.

### Example 3

### (Synthesis of Polyester Polyol Composition (3))

A polymerization vessel equipped with a reflux condenser, a water separator, a nitrogen gas inlet tube, a thermometer and a stirrer was charged with 399.88 parts of glycerol and 590.17 parts of sebacic acid, and a dehydration reaction was performed at 180° C. After the reaction was confirmed to have proceeded to an extent such that the acid value of the reaction product was not more than 3, 122.58 parts of ricinoleic acid (CO-FA manufactured by ITOH OIL CHEMICALS CO., LTD.) was added and a dehydration reaction was conducted at 180° C. When the acid value of the reaction product was confirmed to be not more than 2, the reaction product was withdrawn from the polymerization vessel and was cooled to give a polyester polyol composition (3).

The polyester polyol had a hydroxyl value of 396 mg KOH/g, an acid value of 1.4 mg KOH/g, a viscosity of 19800 mPa·s, and a functionality of 5. The condensation yielded 112.63 parts of water.

### Example 4

### (Synthesis of Polyester Polyol Composition (4))

A polymerization vessel equipped with a reflux condenser, a water separator, a nitrogen gas inlet tube, a thermometer and a stirrer was charged with 344.32 parts of glycerol, 397.01 parts of sebacic acid and 350.92 parts of ricinoleic acid (CO-FA manufactured by ITOH OIL CHEMICALS CO., LTD.), and a dehydration reaction was performed at 180° C. When the acid value of the reaction product was confirmed to be not more than 2, the reaction product was withdrawn from the polymerization vessel and was cooled to give a polyester polyol composition (4).

The polyester polyol had a hydroxyl value of 371 mg KOH/g, an acid value of 1.3 mg KOH/g, a viscosity of 6000 mPa·s, and a functionality of 4. The condensation yielded 92.25 parts of water.

### Example 5

### (Synthesis of Polyester Polyol Composition (5))

A polymerization vessel equipped with a reflux condenser, a water separator, a nitrogen gas inlet tube, a thermometer and a stirrer was charged with 272.84 parts of glycerol and 239.69 parts of sebacic acid, and a dehydration reaction was performed at 180° C. After the reaction was confirmed to have proceeded to an extent such that the acid value of the reaction product was not more than 3, 564.84 parts of ricinoleic acid (CO-FA manufactured by ITOH OIL CHEMICALS CO., LTD.) was added and a dehydration reaction was conducted at 180° C. When the acid value of the reaction product was confirmed to be not more than 2, the reaction product was withdrawn from the polymerization vessel and was cooled to give a polyester polyol composition (5).

The polyester polyol had a hydroxyl value of 334 mg KOH/g, an acid value of 1.2 mg KOH/g, a viscosity of 3000 mPa·s, and a functionality of 3.5. The condensation yielded 77.37 parts of water.

### Example 6

### (Synthesis of Polyester Polyol Composition (6))

A polymerization vessel equipped with a reflux condenser, a water separator, a nitrogen gas inlet tube, a thermometer and a stirrer was charged with 301.2 parts of glycerol and 531.71 parts of azelaic acid, and a dehydration reaction was performed at 180° C. After the reaction was confirmed to have proceeded to an extent such that the acid value of the reaction product was not more than 3, 286.43 parts of ricinoleic acid (CO-FA manufactured by ITOH OIL CHEMICALS CO., LTD.) was added and a dehydration reaction was conducted at 180° C. When the acid value of the reaction product was confirmed to be not more than 2, the reaction product was withdrawn from the polymerization vessel and was cooled to give a polyester polyol composition (6).

The polyester polyol had a hydroxyl value of 484 mg KOH/g, an acid value of 0.4 mg KOH/g, a viscosity of 6670 mPa·s, and a functionality of 4. The condensation yielded 119.34 parts of water.

### Comparative Example 1

### (Synthesis of Polyester Polyol Composition (C1))

A polymerization vessel equipped with a reflux condenser, a, water separator, a nitrogen gas inlet tube, a thermometer and a stirrer was charged with 174.48 parts of glycerol and 184.89 parts of sebacic acid, and a dehydration reaction was performed at 180° C. After the reaction was confirmed to have proceeded to an extent such that the acid value of the reaction product was not more than 3, 694.66 parts of ricinoleic acid (CO-FA manufactured by ITOH OIL CHEMICALS CO., LTD.) was added and a dehydration reaction was conducted at 180° C. When the acid value of the reaction product was confirmed to be not more than 2, the reaction product was withdrawn from the polymerization vessel and was cooled to give a polyester polyol composition (C1).

The polyester polyol had a hydroxyl value of 222 mg KOH/g, an acid value of 0.3 mg KOH/g, a viscosity of 2000 mPa·s, and a functionality of 3. The condensation yielded 54.03 parts of water.

### Comparative Example 2

### (Synthesis of Polyester Polyol Composition (C2))

A polymerization vessel equipped with a reflux condenser, a water separator, a nitrogen gas inlet tube, thermometer and a stirrer was charged with 283.1 parts of glycerol and 310.8 parts of sebacic acid, and a dehydration reaction was performed at 180° C. After the reaction was confirmed to have proceeded to an extent such that the acid value of the reaction product was not more than 3, 451.2 parts of lactic acid (a 90% aqueous solution manufactured by Wako Pure Chemical Industries, Ltd.) was added and a dehydration reaction was conducted at 180° C. When the acid value of the reaction product was confirmed to be not more than 2, the reaction product was withdrawn from the polymerization vessel and was cooled to give a polyester polyol composition (C2).

The polyester polyol had a hydroxyl value of 380 mg KOH/g, an acid value of 0.7 mg KOH/g, a viscosity of 26000 mPa·s, and a functionality of 4. The condensation yielded 136.6 parts of water.

### Example 7

### (Production of Rigid Polyurethane Foam (1) (Foaming Example 1))

A resin premix was prepared by mixing 100.0 parts of the polyester polyol composition (1) from EXAMPLE 1, 6.0 parts of water as a foaming agent, 1.2 parts of a dipropylene glycol solution of triethylene diamine (triethylene diamine: 33%) (hereinafter, also 33 LV) and 0.3 part of a dipropylene glycol solution of bis(2-dimethylaminoethyl)ether (bis(2-dimethylaminoethyl)ether: 70%) (hereinafter, also A-1) as catalysts, and 2.0 parts of SZ-1642 manufactured by Toray Dow Corning Silicone Co., Ltd. as a foam stabilizer.

The resin premix was temperature-controlled at 40° C., and 226.2 parts of polymeric MDI, M-200 manufactured by MITSUI CHEMICALS POLYURETHANES INC. (NCO/H=1.10),was added thereto. The mixture was immediately stirred with a stirrer for 15 seconds. Thereafter, 395 g of the mixture liquid was poured into a closed mold 330×330×80 mm in size that had been warmed at 50° C., and it was cured for 20 minutes. After the completion of the curing, the foam was collected from the mold and was stored at room temperature overnight. The foam was then cut into pieces for the measurement of core density, compressive strength, flexural strength and dimensional stability.

### Example 8

### (Production of Rigid Polyurethane Foam (2) (Foaming Example 2))

A resin premix was prepared by mixing 100.0 parts of the polyester polyol composition (2) from EXAMPLE 2, 6.0 parts of water as a foaming agent, 1.2 -parts of a dipropylene glycol solution of triethylene diamine (triethylene diamine: 33%) (hereinafter, also 33 LV) and 0.3 part of a dipropylene glycol solution of bis(2-dimethylaminoethyl)ether (bis(2-dimethylaminoethyl)ether: 70%) (hereinafter, also A-1) as catalysts, and 1.5 parts of SZ-1642 manufactured by Toray Dow Corning Silicone Co., Ltd. and 1.0 part of X-20-1328 manufactured by Shin-Etsu Chemical Co., Ltd. as foam stabilizers.

The resin premix was temperature-controlled at 40° C., and 225.3 parts of polymeric MDI, M-200 manufactured by MITSUI CHEMICALS POLYURETHANES INC. (NCO/H=1.25), was added thereto. The mixture was immediately stirred with a stirrer for 15 seconds. Thereafter, 395 g of the mixture liquid was poured into a closed mold 330×330×80 mm in size that had been warmed at 50° C., and it was cured for 20 minutes. After the completion of the curing, the foam was collected from the mold and was stored at room temperature overnight. The foam was then cut into pieces for the measurement of core density, compressive strength, flexural strength and dimensional stability.

### Example 9

### (Production of Rigid Polyurethane Foam (3) (Foaming Example 3))

A resin premix was prepared by mixing 100.0 parts of the polyester polyol composition (5) from EXAMPLE 5, 6.0 parts of water as a foaming agent, 1.2 parts of a dipropylene glycol solution of triethylene diamine (triethylene diamine: 33%) (hereinafter, also 33 LV) and 0.3 part of a dipropylene glycol solution of bis(2-dimethylaminoethyl)ether (bis(2-dimethylaminoethyl)ether: 70%) (hereinafter, also A-1) as catalysts, and 2.0 parts of SZ-1642 manufactured by Toray Dow Corning Silicone Co., Ltd. as a foam stabilizer.

The resin premix was temperature-controlled at 40° C., and 224.5 parts of polymeric MDI, M-200 manufactured by MITSUI CHEMICALS POLYURETHANES INC. (NCO/H=1.33), was added thereto. The mixture was immediately stirred with a stirrer for 15 seconds. Thereafter, 495 g of the mixture liquid was poured into a closed mold 330×330×80 mm in size that had been warmed at 50° C., and it was cured for 20 minutes. After the completion of the curing, the foam was collected from the mold and was stored at room temperature overnight. The foam was then cut into pieces for the measurement of core density, compressive strength, flexural strength and dimensional stability.

### Example 10

### (Production of Rigid Polyurethane Foam (4) (Foaming Example 4))

A resin premix was prepared by mixing 100.0 parts of the polyester polyol composition (5) from EXAMPLE 5, 6.0 parts of water as a foaming agent, 1.2 parts of a dipropylene glycol solution of triethylene diamine (triethylene diamine: 33%) (hereinafter, also 33 LV) and 0.3 part of a dipropylene glycol solution of bis(2-dimethylaminoethyl)ether (bis(2-dimethylaminoethyl)ether: 70%) (hereinafter, also A-1) as catalysts, and 2.0 parts of SZ-1642 manufactured by Toray Dow Corning Silicone Co., Ltd. as a foam stabilizer.

The resin premix was temperature-controlled at 40° C., and 224.5 parts of polymeric MDI, M-200 manufactured by MITSUI CHEMICALS POLYURETHANES INC. (NCO/H=1.33), was added thereto. The mixture was immediately stirred with a stirrer for 15 seconds. Thereafter, 395 g of the mixture liquid was poured into a closed mold 330×330×80 mm in size that had been warmed at 50° C., and it was cured for 20 minutes. After the completion of the curing, the foam was collected from the mold and was stored at room temperature overnight. The foam was then cut into pieces for the measurement of core density, compressive strength, flexural strength and dimensional stability.

### Comparative Example 3

### (Production of Rigid Polyurethane Foam (C1) (Foaming Example C1))

A resin premix was prepared by mixing 100.0 parts of the polyester polyol composition (C1) from COMPARATIVE EXAMPLE 1, 6.0 parts of water as a foaming agent, 1.2 parts of a dipropylene glycol solution of triethylene diamine (triethylene diamine: 33%) (hereinafter, also 33 LV) and 0.3 part of a dipropylene glycol solution of bis(2-dimethylaminoethyl)ether (bis(2-dimethylaminoethyl)ether: 70%) (hereinafter, also A-1) as catalysts, and 2.0 parts of SZ-1642 manufactured by Toray Dow Corning Silicone Co., Ltd. as a foam stabilizer.

The resin premix was temperature-controlled at 40° C., and 189.0 parts of polymeric MDI, M-200 manufactured by MITSUI CHEMICALS POLYURETHANES INC. (NCO/H=1.33), was added thereto. The mixture was immediately stirred with a stirrer for 15 seconds. Thereafter, 395 g of the mixture liquid was poured into a closed mold 330×330×80 mm in size that had been warmed at 50° C., and it was cured for 20 minutes. After the completion of the curing, the foam was collected from the mold and was stored at room temperature overnight. The foam was then cut into pieces for the measurement of properties.

The pieces shrank immediately after they were cut, and the measurements of core density, compressive strength, flexural strength and dimensional stability were infeasible.

The results of EXAMPLES 7 to 10 and COMPARATIVE EXAMPLE 3 are set forth in Table 1.

TABLE 1

| | | Ex. 7 | Ex. 8 | Ex. 9 | Ex. 10 | Comp. Ex. 3 |
|---|---|---|---|---|---|---|
| Polyester | Ex. 1 | 100.0 | | | | |
| polyol | Ex. 2 | | 100.0 | | | |
| composition | Ex. 5 | | | 100.0 | 100.0 | |
| | Comp. Ex. 1 | | | | | 100.0 |
| H₂O | | 6.0 | 6.0 | 6.0 | 6.0 | 6.0 |
| 33LV | | 1.2 | 1.2 | 1.2 | 1.2 | 1.2 |
| A-1 | | 0.3 | 0.3 | 0.3 | 0.3 | 0.3 |
| SZ-1642 | | 2.0 | 1.5 | 2.0 | 2.0 | 2.0 |
| X-20-1328 | | | 1.0 | | | |
| M-200 | | 226.2 | 225.3 | 224.5 | 224.5 | 189.0 |
| Mold temperature | (° C.) | 50.0 | 50.0 | 50.0 | 50.0 | 50.0 |
| Mold release time | (min) | 20.0 | 20.0 | 20.0 | 20.0 | 20.0 |
| Resin temperature | (° C.) | 40.0 | 40.0 | 40.0 | 40.0 | 40.0 |

| Core density (kg/m³) | | | 37.9 | 37.6 | 45.0 | 38.0 | Measurements |
|---|---|---|---|---|---|---|---|
| Compressive | strength | (kPa) | 207.1 | 211.6 | 271.6 | 172.2 | were |
| Flexural | strength | (kPa) | 339.0 | 143.9 | 491.1 | 369.5 | infeasible |
| Dimensional | High | (%) | −0.6 | −0.8 | −0.4 | −1.0 | because |
| change | temperature | | | | | | samples |
| | Humid | (%) | −1.1 | −0.3 | −0.4 | −2.2 | shrank |
| | heating | | | | | | immediately |
| | Low | (%) | −0.8 | −0.5 | −0.3 | −0.4 | after |
| | temperature | | | | | | cutting. |

| | | | | |
|---|---|---|---|---|
| Closed cell percentage (%) | 91.0 | 88.7 | 90.7 | 87.8 |

High temperature: Standing at 70° C. for 72 hours.
Low temperature: Standing at −30° C. for 72 hours.
Humid heating: Standing at 70° C. and 95% humidity for 72 hours.

### INDUSTRIAL APPLICABILITY

The polyol compositions for rigid polyurethane foams are usable as thermal insulating materials in building panels, refrigerators, freezers and pipes, and as structural supports in housings and vehicles.

1. A polyester polyol composition obtained by:
a step (I) in which at least one compound (A) selected from the group consisting of dibasic acids, alkyl esters formed between a C1-6 monohydric saturated alcohol and a dibasic acid, and alkenyl esters formed between a C3-6 monohydric unsaturated alcohol with one carbon-carbon double bond and a dibasic acid, is reacted with a trihydric or higher polyhydric alcohol (a compound (B)); and
a step (II) in which the reaction mixture from the step (I) is reacted with at least one compound (C) selected from the group consisting of hydroxycarboxylic acids, alkyl esters formed between a C1-6 monohydric saturated alcohol and a hydroxycarboxylic acid, and alkenyl esters formed between a C3-6 monohydric unsaturated alcohol with one carbon-carbon double bond and a hydroxycarboxylic acid;
at least one compound (C) being a compound (C′) selected from the group consisting of ricinoleic acid, alkyl esters formed between a C1-6 monohydric saturated alcohol and ricinoleic acid, alkenyl esters formed between a C3-6 monohydric unsaturated alcohol with one carbon-carbon double bond and ricinoleic acid, 12-hydroxystearic acid, alkyl esters formed between a C1-6 monohydric saturated alcohol and 12-hydroxystearic acid, and alkenyl esters formed between a C3-6 monohydric unsaturated alcohol with one carbon-carbon double bond and 12-hydroxystearic acid;
the polyester polyol composition having a hydroxyl value in the range of 250 to 550 mg KOH/g and an average functionality of 3 to 8.

2. A polyester polyol composition obtained by:
a step (1) of reacting at least one compound (A) selected from the group consisting of dibasic acids, alkyl esters formed between a C1-6 monohydric saturated alcohol and a dibasic acid, and alkenyl esters formed between a C3-6 monohydric unsaturated alcohol with one carbon-carbon double bond and a dibasic acid;
a trihydric or higher polyhydric alcohol (a compound (B)); and
at least one compound (C) selected from the group consisting of hydroxycarboxylic acids, alkyl esters formed between a C1-6 monohydric saturated alcohol and a hydroxycarboxylic acid, and alkenyl esters formed between a C3-6 monohydric unsaturated alcohol with one carbon-carbon double bond and a hydroxycarboxylic acid;
at least one compound (C) being a compound (C′) selected from the group consisting of ricinoleic acid, alkyl esters formed between a C1-6 monohydric saturated alcohol and ricinoleic acid, alkenyl esters formed between a C3-6 monohydric unsaturated alcohol with one carbon-carbon double bond and ricinoleic acid, 12-hydroxystearic acid, alkyl esters formed between a C1-6 monohydric saturated alcohol and 12-hydroxystearic acid, and alkenyl esters formed between a C3-6 monohydric unsaturated alcohol with one carbon-carbon double bond and 12-hydroxystearic acid;
the polyester polyol composition having a hydroxyl value in the range of 250 to 550 mg KOH/g and an average functionality of 3 to 8.

3. The polyester polyol composition according to claim 1 or 2, which is obtained using 2 to 70 parts by weight of the compound (A), 10 to 85 parts by weight of the compound (B), and 5 to 85 parts by weight of the compound (C) (the total of the compounds (A), (B) and (C) is 100 parts by weight).

4. The polyester polyol composition according to any one of claims 1 to 3, wherein the compound (C′) accounts for 10 to 100 wt % of 100 wt % of the compound(s) (C).

5. The polyester polyol composition according to any one of claims 1 to 4, wherein the compound (A), the compound (B) and the compound (C) are each a plant-derived compound.

6. A composition for rigid polyurethane foams comprising:
the polyester polyol composition of any one of claims 1 to 5;
a polyisocyanate;
at least one foaming agent selected from the group consisting of water and hydrocarbon compounds; and
a catalyst.

7. A rigid polyurethane foam formed from the composition for rigid polyurethane foams of claim 6.
